(19) United States
(12) Patent Application Publication
(10) Pub. No.: US 2011/0076694 A1
(43) Pub. Date: Mar. 31, 2011

(54) METHOD OF ASSESSING MAMMALIAN EMBRYO QUALITY

(76) Inventors: Gerald M. Kidder, London (CA); Hong-Xing Wang, Boston, MA (US); Dan Tong, Boston, MA (US); Francis R. Tekpetey, London (CA)

(21) Appl. No.: 12/920,670

(22) PCT Filed: Mar. 5, 2009
(86) PCT No.: PCT/CA09/00266

§ 371 Date: Nov. 25, 2010

Related U.S. Application Data
(60) Provisional application No. 61/064,421, filed on Mar. 5, 2008.

(51) Int. Cl. G01N 33/53; C12Q 1/02

(52) U.S. Cl. 435/7.9; 435/29; 435/7.1

(57) ABSTRACT

A method of assessing the health of each oocyte or embryo of a mammal from a plurality of oocytes/embryos of the mammal is provided. The method comprises the steps of: measuring gap junctional coupling strength or connexin43 expression of follicular cells associated with each oocyte; and identifying the gap junctional coupling strength/connexin43 expression of follicular cells associated with each oocyte, wherein the greater the coupling strength/connexin43 expression of follicular cells associated with an oocyte relative to the coupling strength/connexin43 expression of follicular cells associated with each other oocyte, the healthier the oocyte and the embryo resulting from that oocyte. Kits for conducting these methods are also provided.

### FIELD OF THE INVENTION

The present invention relates to a method of assessing the quality of an oocyte or embryo arising from the oocyte relative to a plurality of oocytes/embryos. In particular, the invention relates to the determination of certain properties of follicular cells as an indicator of both oocyte and embryo quality.

### BACKGROUND OF THE INVENTION

Gap junctions are clusters of intercellular membrane channels that allow direct exchange of small molecules, including nutrients, metabolites, and second messengers, between cells. An individual gap junction channel is formed when two hemichannels, one from each cell, dock end-to-end to form an intercellular channel. Hemichannels are called connexons and each is a hexamer of subunits called connexins (Cx). In mammals, connexins are encoded by a multigene family with 20 or more members. Individual connexins are distinguished by their sizes: Connexin43 (C×43), for example, is a ˜43 kD protein whereas C×40 is a ˜40 kD protein. Gap junction channels composed of different connexins differ in their permeability to specific signaling molecules, properties that are assumed to underlie the physiological roles played by gap junctions in different cell types.

Ovarian folliculogenesis requires complex regulatory mechanisms involving both endocrine and intra-ovarian signaling pathways. In developing follicles, gap junctions couple the growing oocyte and its surrounding granulosa cells into a functional syncytium allowing amino acids, glucose metabolites, and nucleotides to be transferred to the oocyte [1]. In addition, signals that regulate meiotic maturation of fully grown oocytes (including Ca²⁺ and cAMP) are thought to pass through the oocyte-granulosa cell gap junctions. Recent findings from gene expression studies in several species and gene targeting in mice have implicated gap junctional intercellular communication (GJIC) in follicular development and have suggested its involvement in female infertility.

Given the foregoing, it would be desirable to determine the role that connexins and gap junctional coupling play in mammalian oogenesis in order to develop methodology that may improve assisted reproductive technologies such as in vitro fertilization techniques.

### SUMMARY OF THE INVENTION

It has now been found that gap junctional coupling strength and level of connexin43 (C×43) expression in mammalian follicular cells may each be used as determinants of the quality or health of oocytes associated therewith, as well as embryos resulting from the oocytes.

Thus, in one aspect of the present invention, a method of assessing the health of each oocyte, or an embryo that may result therefrom, from a plurality of oocytes of a mammal is provided comprising the steps of:
i) measuring gap junctional coupling strength of follicular cells associated with each oocyte; and
ii) identifying the gap junctional coupling strength associated with each oocyte, wherein the greater the coupling strength of an oocyte relative to the coupling strength of each other oocyte, the healthier the oocyte and an embryo resulting from that oocyte.

In another aspect, a method of assessing the health of each oocyte, or an embryo that may result therefrom, from a plurality of oocytes of a mammal is provided, comprising the step of determining the expression level of connexin43 in a sample of follicular cells associated with each oocyte, wherein the greater the expression level of connexin43 in a sample of follicular cells relative to the connexin43 expression level obtained for each other sample of follicular cells, the healthier the oocyte and the embryo that may result therefrom.

In another aspect, a kit useful to assess the health of oocytes of a mammal, or embryos resulting therefrom, is provided comprising:
i) a dye-delivery means adapted to deliver a controlled amount of dye to a single follicular cell of a sample of follicular cells associated with an oocyte or embryo, wherein the dye-delivery means includes a suitable dye for injection into the follicular cell; and
ii) an indication that the greater the dye uptake of a sample of follicular cells associated with an oocyte/embryo of a mammal relative to the dye uptake of samples of follicular cells associated with other oocytes/embryos for the mammal, the healthier the oocyte or embryo.

In a further aspect, a kit useful to assess the health of oocytes of a mammal, or embryos resulting therefrom, is provided comprising:
i) a solid support to which is bound a first connexin43-specific reactant useful to immobilize connexin43 thereto from a follicular cell sample; and
ii) an indicator suitable for use to detect bound connexin43.

In a further aspect, a method of assessing the health of an oocyte of a mammal, or an embryo that may result therefrom, in comparison to a plurality of oocytes from other like mammals, is provided comprising the steps of:
i) measuring gap junctional coupling strength of follicular cells associated with each oocyte; and
ii) identifying the gap junctional coupling strength associated with each oocyte, wherein the greater the coupling strength of the oocyte relative to the coupling strength of each other oocyte, the healthier the oocyte and the embryo resulting from that oocyte.

In yet a further aspect, a method of assessing the health of an oocyte of a mammal, or an embryo that may result therefrom, in comparison with a plurality of oocytes of other like mammals, is provided comprising the step of determining the expression level of connexin43 in a sample of follicular cells associated with each oocyte, wherein the greater the expression level of connexin43 associated with the oocyte relative to the connexin43 expression level associated with each other oocyte, the healthier the oocyte and the embryo that may result therefrom.

These and other aspects of the invention are described herein by reference to the following drawings.

### BRIEF DESCRIPTION OF THE DRAWINGS

FIG. 1 illustrates quantification of C×43 in human follicular cells from different patients by western blotting (A) and graphically in comparison to GAPDH and vimentin as internal controls (B);

FIG. 2 graphically illustrates the variation of gap junctional conductance among follicular cells from different patients;

FIG. 3 graphically illustrates that C×43 level is related to the strength of gap junctional conductance in human follicular cells by a showing that gap junctional conductance is significantly greater in follicular cell samples having a relative C×43 level greater than the population mean (A), and that C×43 level is significantly greater in follicular cell samples having a conductance level greater than the population mean (B);

FIG. 4 graphically illustrates the relationship between C×43 level and clinical data including MII rate, fertilization rate, transferable embryo rate and implantation rate using vimentin as the standard (A) and GAPDH as a standard (B), and also illustrates the relationship between C×43 level and pregnancy outcome (C);

FIG. 5 graphically illustrates the relationship between gap junctional conductance and clinical data including MII rate, fertilization rate, transferable embryo rate, implantation rate and pregnancy rate (the ratio of number of pregnant patients to number of total patients in each group) (A), and a comparison of conductance level between pregnant patients and non-pregnant patients (B);

FIG. 6 illustrates typical results of a dye transfer assay determined by order of dye transfer (A) and by the proportion of cells dyed (B) for the determination of gap junctional coupling strength among follicular cells;

FIG. 7 graphically illustrates the correlation of coupling strength of individual follicles, as determined using a dye transfer assay, with the % embryos transferred and the % of resulting pregnancy;

FIG. 8 illustrates the coupling strength of multiple follicles of individual patients; and

FIG. 9 graphically illustrates the relationship between mean coupling strength of follicles that gave rise to transferred embryos and pregnancy rate.

### DETAILED DESCRIPTION OF THE INVENTION

A method of assessing the health of an oocyte of a mammal in comparison to a plurality of oocytes from the same mammal, or the health of an embryo arising from an oocyte of a mammal in comparison to a plurality of embryos arising from the oocytes of the mammal, is provided comprising measuring the gap junctional coupling strength of follicular cells associated with each oocyte and determining a gap junctional coupling strength value associated with each oocyte. The greater the coupling strength value associated with an oocyte relative to the coupling strength values obtained for each oocyte, the healthier the oocyte and the healthier the embryo arising from that oocyte.

As one of skill in the art will appreciate, the term “embryo” refers to a fertilized oocyte. An oocyte, ovocyte or egg is a female gametocyte or germ cell found within follicles in the ovary of a female mammal. A follicle is a sphere of cells within which an oocyte develops.

The term “mammal” is used herein to encompass humans as well as animals which are reproductively similar to humans, including for example, livestock such as horses, cows, sheep and goats, in which the method is useful to increase the success of commercial breeding, and wild animals, for example, to aid in captive breeding programs of endangered species. The term “reproductively similar” refers to the similar involvement of follicular cells in supporting the growth and development of each individual oocyte.

The term “health” with respect to an oocyte or embryo is used interchangeably herein with the term “quality” to denote the physical condition of the oocyte or embryo and is indicative of the utility thereof for use in treatments such as assisted reproductive technologies, as well as for the research and development of the treatment of disease, for example, regenerative therapies.

The present method is useful to assess oocyte and embryo health or quality by a comparison of the gap junctional coupling strength of the cells of different follicles from the same mammalian subject to obtain a relative assessment of the health of the oocyte (and subsequent embryo) derived from or associated with each follicle. The term “follicular cells” as it used herein refers to cells of a follicle including, for example, but not limited to cumulus granulosa cells. In addition, the terms “derived from” and “associated with” with respect to an oocyte refer to the follicle within which the oocyte develops. By assessing the quality of oocytes based on follicular cell gap junctional coupling strength, the healthiest oocytes from the follicles of a given mammalian subject, e.g. an oocyte from a follicle that has the strongest coupling strength, may be utilized as described herein as opposed to oocytes from follicles providing a weaker coupling strength.

In one aspect of the invention, gap junctional coupling strength between cells within a follicle, e.g. the nature of channels providing communication between the cells of the follicle, as an indicator of the health of the oocyte from the follicle, and on fertilization of the oocyte, an indicator of embryo health, may be determined by measuring the electrical conductance between the cells of the follicle yielding the oocyte. Electrical conductance may be measured using the patch clamp technique well-established in the art. Briefly, a glass patch pipette is pressed against the membrane of a selected follicular cell. The pipette is filled with an electrolyte solution allowing a depolarization voltage pulse to generate a transient capacitive current. The strength of the current indicates the strength of gap junctional conductance between the follicular cells.

Gap junctional coupling strength of follicular cells may also be determined using a dye transfer assay. Generally, this assay is conducted by injecting a dye solution into a single cell of a colony of follicular cells (e.g. resulting from the overnight culturing of cumulus cells from individual follicles) with an appropriate delivery means, such as a beveled micropipette, and measuring the extent of transfer of the dye to other cells in the cell cluster. A concentration of dye in the range of about 1-5%, depending on the dye chosen, is injected into a cell for a determination of coupling strength via dye transfer. For consistency, dye injection time is desirably maintained from follicle to follicle, for example, an injection time of about 1 minute is appropriate. The dye will usually fill the cell without the need for additional pressure, however, as one of skill in the art will appreciate, a slight pressure (e.g. 15-20 kPa) can be applied to encourage the dye to fill the cell.

Dyes suitable for use in this assay have a mass that is generally less than approximately 800 Daltons and are preferably are readily detectable, e.g. by fluorescence. Thus, dyes suitable for this purpose include, but are not limited to, acridine dyes, arylmethane dyes, azo dyes, cyanine dyes, diazonium dyes, nitro dyes, nitroso dyes, quinone-imine dyes including azin dyes such as eurodin and safranin, indophenol dyes, oxazin and oxazone dyes, thiazin and thiazole dyes and xanthene dyes including fluorene dyes such as rhodamine, e.g. Rhodamine 6G and sulforhodamine 101 acid chloride. In another embodiment, dye transfer may be effected by electrostatic injection of ionized dyes, e.g. eosin, malachite green, methylene blue and basic/acidic fuchsin, using established techniques.

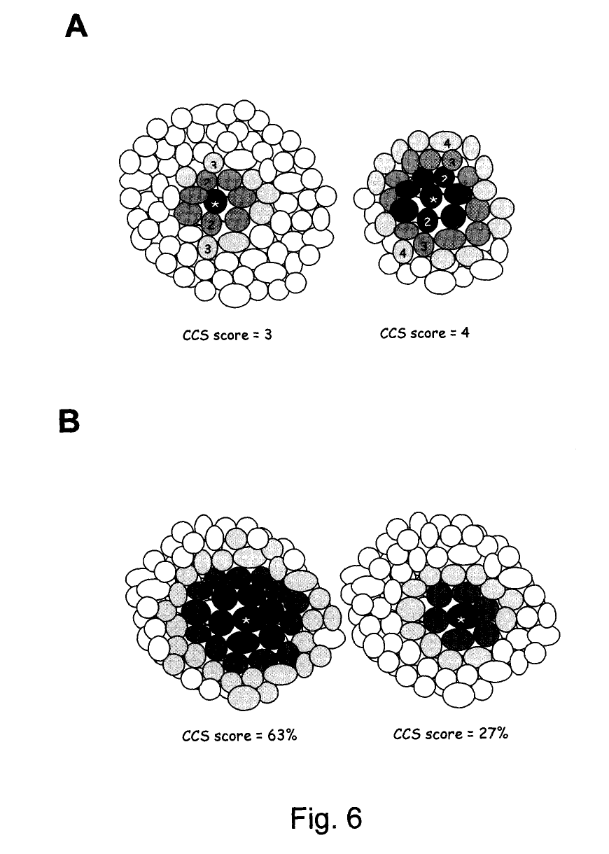

Dye transfer among cells of a follicle is measured to determine coupling strength after a standard amount of time, e.g. about 2 minutes, following dye injection using established techniques, including determining the order of dye transfer, e.g. the number of cells away from the injected cell in which dye can be detected. This method is particularly useful when colony size differs between individual follicles. FIG. 6(A) illustrates 3rd and 4^{th} order dye transfer, respectively. Alternatively, the strength of coupling can be determined by the proportion of dye transfer, e.g. the per cent of cells in a cluster that received the dye from the injected cell. FIG. 6(B) illustrates coupling strength scores of 63% and 27%, respectively. This method is useful where cell colonies arising from individual follicles are of uniform size. The greater the strength of dye transfer, the greater the gap junctional coupling strength.

Having determined the gap junctional coupling strength of multiple follicles of the same mammal, it can then be determined which oocytes from the mammal are the healthiest. In accordance with the invention, the greater the gap junctional coupling strength, the greater the health or quality of the oocyte, and the greater the health of the embryo arising from the oocyte.

In addition, it has also been found that the expression level of connexin43 (C×43) in cells of a follicle is a determinant of coupling strength. The greater the expression level of C×43, the greater the coupling strength. Accordingly, in another aspect of the invention, the expression level of C×43 in follicular cells may also be used to determine the health or quality of oocytes/embryos. The level of C×43 expression may be determined at the protein level, e.g. the amount of C×43 protein in follicular cells, or at the nucleic acid level, e.g. the amount of C×43-encoding mRNA within the follicular cells, using well-established techniques.

C×43 protein in cells from individual follicles of the same mammal may be identified and quantified using techniques in the art including, for example, enzyme-linked immunosorbent assay (ELISA) or gel electrophoresis (e.g. western blot). Within a cohort of follicles from a mammalian subject, oocytes of follicles having the highest C×43 level relative to one or more internal standards such as vimentin or GAPDH are the healthiest oocytes of the cohort and, thus, the most suitable for use, for example, to generate an embryo.

Alternatively, the identification and quantification of C×43-encoding mRNA from individual follicles of a mammal may also be determined using well-established techniques such as RT-PCR or northern blot. Within a cohort of follicles from a mammalian subject, oocytes from follicles having the highest C×43 mRNA levels relative to one or more internal standards such as vimentin or GAPDH mRNA are the healthiest of the cohort and, thus, the most suitable for use, for example, to generate an embryo.

The determination of oocyte health via C×43 determination may be conducted alone or in combination with the determination of other follicular cell proteins such as, for example, cytochrome P450 aromatase (CYP1 9A1), cell division cycle 42 (CDC42), 3-β-hydroxysteroid dehydrogenase 1 (3(3βSD1), serpin peptidase inhibitor Glade E member 2 (SERPINE 2), adrenodoxin (ADX), 11 beta-hydroxy-steroid dehydrogenase (11βHSD) and a relaxin.

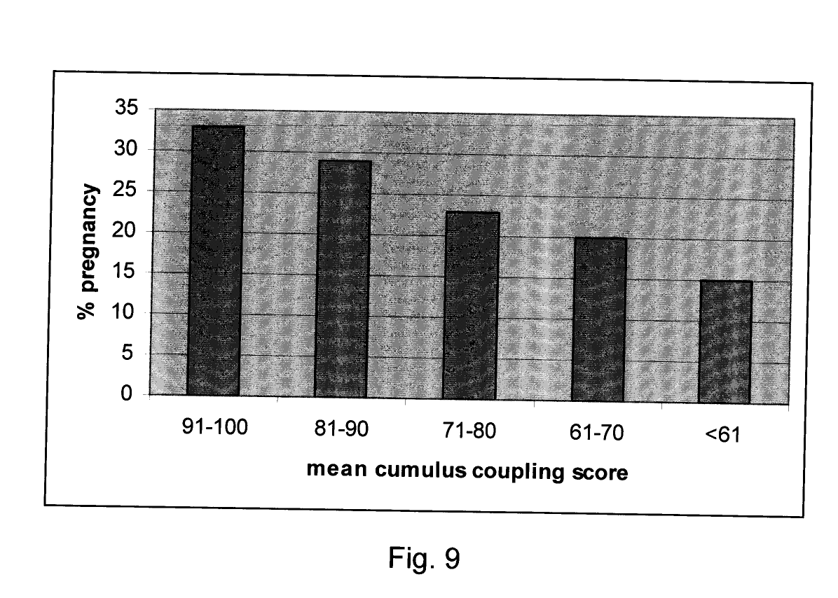

In the event that only one or a small number of oocytes (or embryos) are available from a given mammalian subject, the present methodology may also be utilized to assess the health/quality thereof by comparing the gap junctional coupling strength of the follicular cells associated with a single oocyte with the gap junctional coupling strength of follicular cells associated with oocytes from other like mammalian subjects (e.g. comparing human oocytes against human oocytes), or by comparing connexin43 expression of follicular cells associated with the single oocyte with the connexin43 expression of follicular cells associated with oocytes of other like mammalian subjects. A gap junctional coupling strength or connexin43 value that is greater than the median value obtained for oocytes of other mammalian subjects may be indicative of an oocyte that may be suitable for use, for example, to generate an embryo, or may be indicative of an embryo that may be suitable for use in assisted reproductive technologies. For example, by reference to the coupling strength data in FIG. 9 (Example 3) obtained for a series of patients, a coupling strength value of about 70 for the follicular cells of a single oocyte from a given mammal may be indicative of the utility of that oocyte. As one of skill in the art will appreciate, the utility of this method of determining oocyte/embryo health depends on the comparability of gap coupling strength values and/or connexin43 expression values. These values must be comparable values, e.g. obtained using the same or similar instrumentation under the same or similar conditions, e.g. generated in the same clinical setting.

In connection with a further aspect of the invention, a kit is provided useful to assess oocyte and/or embryo quality via a determination of gap junctional coupling strength in a sample of follicular cells of the oocyte. The kit comprises a dye-delivery means adapted to deliver dye to a single follicular cell. An example of a suitable dye-delivery means is a micropipette with a beveled tip having a diameter in the range of about 0.5 to 1.0 μm. The micropipette may have a length of about 5-8 cm, the body of which may have an internal diameter of about 0.5-0.75 mm and an outer diameter of about 1 mm). The dye-delivery means is filled with a suitable amount of an appropriate dye for injection into a selected cell, for example a dye solution including an amount of dye in the range of about 1-5%, depending on the dye chosen.

The kit may also include a depiction, either textual or by illustration, providing instructions on culturing follicle cells, performing dye injections, photographing the cells following dye injection and interpretation of the results, including how dye transfer strength may be used to assess the health of an oocyte or embryo arising therefrom. Thus, the kit may illustrate different levels of dye transfer strength and indicate that the greater the dye transfer strength of the follicular cells associated with an oocyte/embryo of a mammal relative to the dye transfer strength of follicular cells associated with other oocytes/embryos from the mammal, the healthier the oocyte or embryo.

A kit useful to assess oocyte and/or embryo quality via a determination of connexin43 expression in a sample of follicular cells of the oocyte is also provided. The kit may be based on an ELISA-type platform, for example, and comprise a solid support, such as a microwell plate or beads, to which is bound a first connexin43-specific reactant, such as an antibody directed to a specific part of connexin43, to immobilize connexin43 from a cellular sample. An example of such an antibody is Sigma-Aldrich C6219, which is raised against a synthetic peptide corresponding to the C-terminal segment (amino acids 363-382) of connexin43. The kit will also include an indicator suitable for use to detect bound connexin43, e.g. a detectable label such as an enzyme, examples of which include, but are not limited to, horseradish peroxidase (HRP), alkaline phosphatase (AP), β-galactosidase, acetylcholinesterase and catalase that is capable of yielding a detectable product on reaction with a substrate, which may optionally also be included in the kit. Fluorescent labels may also be used. The indicator is adapted to link to connexin43 (e.g. by association with a second connexin43-specific reactant such as an antibody that is directed to a different part of connexin43 than the first connexin43-specific reactant). A large selection of substrates is available for performing the ELISA. As one of skill in the art will appreciate, useful substrates will depend on the level of detection required and the detection instrumentation used, e.g. spectrophotometer, fluorometer or luminometer. The present kit may also be adapted for the determination of additional follicular cell proteins such as cytochrome P450 aromatase (CYP1 9A1), cell division cycle 42 (CDC42), 3-β-hydroxysteroid dehydrogenase 1 (3βHSD1), serpin peptidase inhibitor Glade E member 2 (SERPINE 2), adrenodoxin (ADX), 11β-hydroxysteroid dehydrogenase (11βHSD) and a relaxin, including on the solid support reactants specific for the additional proteins, and indicators suitable to detect these proteins similar to that discussed to the detection of connexin43.

As one of skill in the art will appreciate, the methods and kit described herein are useful for the determination of both oocyte and embryo health/quality due to the fact that the health of an embryo is dependent, at least to a certain degree, on the health of the oocyte from which it is created and the health of an oocyte is dependent, at least to a certain degree, on the strength of gap junctional coupling within the follicle in which it develops.

Although the present methods and kit are particularly useful to assess the quality or health of an oocyte for subsequent use in assisted reproductive technologies such as in vitro fertilization, and to assess the health of an embryo for implantation into a female recipient, an assessment of the health of an embryo may also be useful for the purpose of developing cell lines therefrom, for example, embryonic stem cell lines, for use in the development of treatments and/or regenerative therapies for a wide range of disease states including, but not limited to, bone loss, broken bones, brain damage due to oxygen starvation, severe burns, cancer, diabetes, Lou Gehrig's disease, heart disease, hepatitis, Huntington's disease, leukemia, lupus, muscular dystrophy, multiple sclerosis, osteoarthritis, Parkinson's disease, spinal cord injuries and stroke.

Embodiments of the invention are described by reference to the following specific example which is not to be construed as limiting.

### Example 1

### Materials and Methods

### Patients

Patients in this study were undergoing treatment in the Reproductive Endocrinology and Infertility Program at the London Health Sciences Centre, London, Ontario, Canada. The study design was approved by the Health Sciences Research Ethics Board of the University of Western Ontario and all patients gave informed consent. Follicular cells were collected from oocytes being prepared for ICSI with day 3 embryo transfer. Clinical data, including mature oocyte rate (MII rate), fertilization rate, transferable rate (% embryos with more than 5 blastomeres and good morphology on day 3), implantation rate (ratio of number of fetuses to number of embryos transferred), and pregnancy outcome (determined by ultrasound 40 days after oocyte retrieval) were obtained by clinical staff, but the research team were blind to these outcomes until all data had been collected for all patients. A total of 115 women donated their follicular cells for this study. All follicular cells from each patient's oocytes were considered as one sample. Eleven samples were used for RT-PCR, 26 samples for immunofluorescence, 81 samples for western blotting, and 42 samples for gap junctional coupling assay (some samples were used for more than one type of analysis). All products for this study were purchased from Invitrogen Canada Inc. (Burlington, ON, Canada) unless otherwise indicated.

### Follicular Cell Culture

Follicular cells were washed twice with culture medium consisting of DMEM/F12 (1:1) supplemented with 10% fetal bovine serum (FBS), 100 units/mL penicillin and 100 μg/mL streptomycin. The cells were grown on glass coverslips treated with 0.358 mg/mL collagen (BD Biosciences, Mississauga, ON) and cultured at 37° C., 5% CO₂ in air for no more than 48 hours.

### Western Blotting

Whole cell proteins were extracted with lysis buffer containing 50 mmol/L Tris-HCl (pH 8.0), 150 mmol/L sodium chloride, 0.02% sodium azide, 100 μg/mL phenylmethylsulfonyl fluoride, 1% NP-40, 0.1% SDS, 1 μg/mL aprotinin and 0.5% sodium deoxycholate. Samples were used for two or three experiments depending on the number of cells obtained from the patient. Proteins were separated by SDS-polyacrylamide gel electrophoresis (SDS-PAGE) on 12% gels and transferred to nitrocellulose membrane (Amersham Pharmacia Biotech, Little Chalfout, Buckinghamshire, England). The membrane was blocked with 5% nonfat milk (w/v) in TBST for 1 h, and subsequently probed with anti-C×43 antibody (1:5,000; Sigma, Oakville, ON) overnight at 4° C. followed by incubation with horseradish peroxidase (HRP)-conjugated secondary antibody (1:5,000; Biolynx Inc., Brockville, ON) for 1 h. Antibody binding was detected by ECL™ Western Blotting Detection Reagent (Amersham Biosciences, Little Chalfout, Buckinghamshire, England). The membrane was then stripped and re-probed with anti-glyceraldehyde-3-phosphate dehydrogenase (GAPDH) antibody (1:400; Chemicon International Inc., Temecula, Calif.) and anti-vimentin antibody (1:500; Sigma) for 1 h respectively then incubated with HRP-conjugated secondary antibody (1:5,000) for detection with the Amersham ECL™ Reagent. The relative intensity of C×43 bands was determined by reference to the GAPDH and vimentin bands, and quantified using Quantity One software (Bio-Rad Laboratories (Canada) Ltd, Mississauga, ON).

### Gap Junctional Conductance Measurement

Single-electrode whole cell patch-clamp recording was used to measure follicular cell membrane capacitance and gap junctional conductance as described [2], the contents of which are incorporated herein by reference, particularly page C291. Briefly, pipettes were made from borosilicate glass capillaries using a two-stage pipette puller (PP-83; Narishige, Tokyo, Japan). The intracellular pipette solution contained 70 mM KCl, 70 mM CsCl, 2 mM EGTA, 4 mM MgCl₂, 5 mM TEA-Cr, and 10 mM HEPES, pH 7.3, and pipettes had a resistance of 3-5 MΩ. Cells on coverslips were transferred to a 2-ml recording chamber mounted on the stage of an inverted microscope (Olympus IMT-2). They were bathed in solution containing 140 mM NaCl, 5.4 mM KCl, 1 mM MgCl₂, 1.8 mM CaCl₂, and 20 mM HEPES, pH 7.4. Voltage clamp for whole-cell recordings was carried out with an Axopatch 200B amplifier (Axon Instruments Inc., Union City, Calif.). Voltage clamping was applied to a single cell in a cluster with 15-20 cells. A depolarization voltage pulse (10 mV, 120-ms duration) was used to generate a transient capacitive current. The peak current and the steady-state current were measured. Currents were high-cut filtered at 10 kHz and digitized at 100 kHz. The experiment was repeated at least four times for every follicular cell sample. The estimated conductance between the patched cell and its surrounding cells was calculated. Data acquisition and analysis were performed using the Digidata 1200A interface and pClamp6 software (Axon Instruments).

### Statistical Analysis

Relative levels of C×43 protein normalized to GAPDH or vimentin were calculated and compared with gap junctional coupling strength as determined by conductance assay. Similarly, relative C×43 levels were compared with pregnancy outcome based on ultrasound. Overall, 35 patients in this study became pregnant and 46 did not, with the age of the former group being 31.2±0.71 and that of the latter group being 34.5±0.69 (mean±SEM). To carry out these comparisons, the patients were divided into two groups based either on whether their mean C×43 or intercellular conductance measurement fell above or below the population mean, or whether they became pregnant. Age of patients was one of factors analyzed in this study, and all patients were divided into three age groups (30 and under, 31-35, 36 and above) to look for any association between C×43 or conductance level and patient age. Statistical analysis (one-way ANOVA) was performed using the Statistical Package for Social Science (SPSS 13.0 for Windows; SPSS Inc., Chicago, Ill.). P<0.05 was considered to be significant.

### Results

### Quantification of C×43

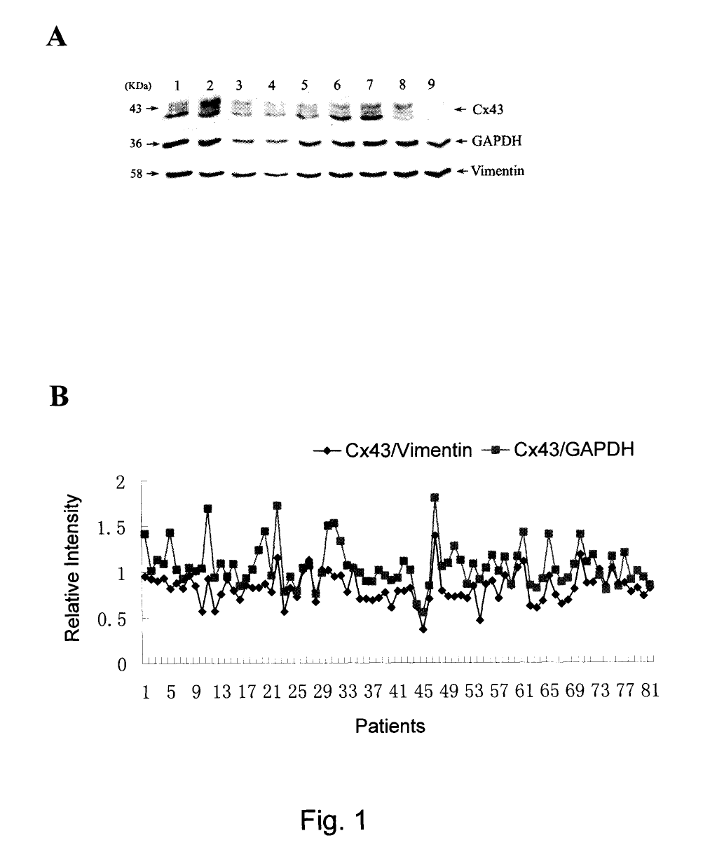

C×43 was detected in all 81 samples tested. A representative western blot is shown as FIG. 1A. The relative amount of C×43 protein was determined by reference to two internal controls, vimentin and GAPDH, revealing variation in C×43 expression level between follicular cells of different patients. The relative C×43 protein levels determined from the two internal controls were fairly consistent between patients (FIG. 1B).

### Quantification of Gap Junctional Conductance

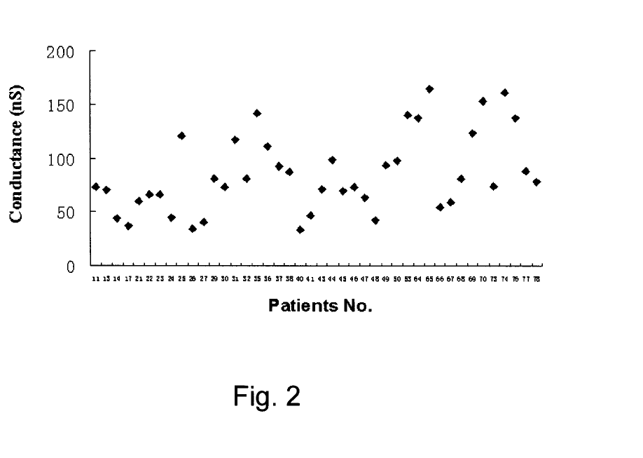

Patch clamp electrophysiology provides a sensitive and quantifiable means of measuring electrical conductance between cells. A 10 mV depolarizing voltage pulse in a voltage-clamped single follicular cell resulted in a current transient characterized by a rapid onset to reach peak current, followed by a rapid decay to steady state current that was almost identical to the holding current. The changes in decay time constant and steady-state current in a cluster of interconnected follicular cells provide a quantitative measure of conductance due to gap junctional coupling of the cells [2]. The estimated conductance was taken as a measure of the total gap junctional conductance between the cells. This conductance varied between patients, although most patients showed conductance above 80 nS (FIG. 2).

### Relation Between C×43 and Gap Junctional Conductance

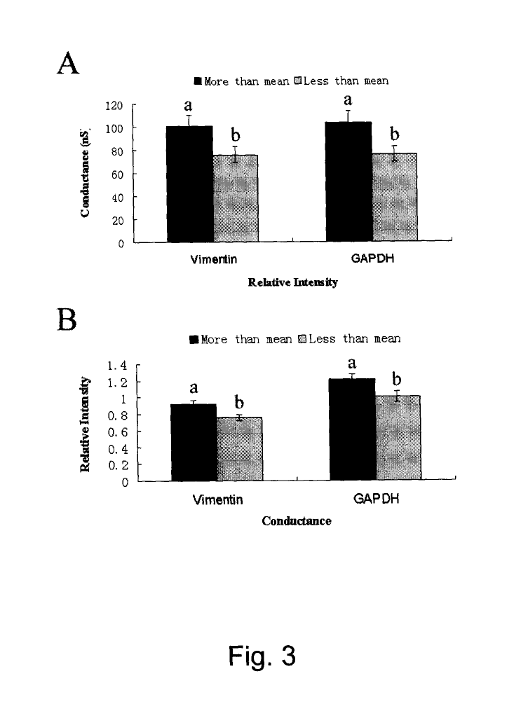

Given that C×43 was the only connexin detected that formed numerous gap junction-like plaques between the follicular cells, it was determined whether or not the level of C×43 is related to the strength of gap junctional coupling. The results confirmed that the level of C×43 is related to the strength of gap junctional coupling by showing that gap junctional conductance is significantly greater in follicular cell samples whose relative level of C×43 protein, indicated by the relative band intensity with either vimentin or GAPDH as internal standard, is greater than the mean of all samples (FIG. 3A). Conversely, the relative level of C×43 is significantly greater in those follicular cell samples whose conductance is greater than the mean of all samples (FIG. 3B).

### Relation Between C×43 or Gap Junctional Conductance and Patient Age

Because patient age is an important factor for pregnancy outcome in IVF treatment, whether an association between age and C×43 or conductance level exists was examined. The results showed that, in the present patient population, neither C×43 nor conductance level differed significantly between age groups. Thus, the mean age of patients in the high C×43 or conductance group was equal to that in the low C×43 or conductance group.

### Relation Between C×43 or Gap Junctional Conductance and Clinical Data

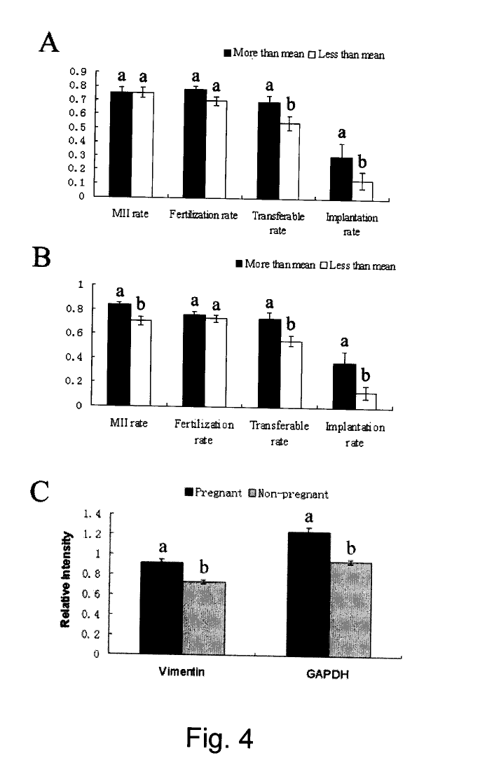

Since C×43 level in follicular cells correlates with gap junctional conductance, the possibility that clinical outcomes from ICSI are related to C×43 level was explored. Patients were partitioned into two groups based on whether their follicular cell C×43 expression was above or below the mean for all patients. Oocytes were evaluated for nuclear maturity and graded as metaphase II (MII), metaphase I, or prophase I. Fertilization was considered have occurred when two clear pronuclei were present after 16-18 h insemination. Embryo transferability was estimated on day 3 post-insemination according to a grading system, with embryos having more than 6 blastomeres and good morphology being considered as transferable. Oocyte maturation (MII) rate and fertilization rate in the high C×43 group were not different from those in the low C×43 group using vimentin as a standard, although the MB rate in the high C×43 group was slightly higher than that in the low C×43 group using GAPDH as the standard (FIG. 4A, B). On the other hand, higher C×43 level was significantly associated with higher transferable rate and implantation rate. Pregnancy outcome (as determined by day 40 ultrasound) was used to partition the 81 patients for which relative C×43 level was determined into two groups, and the mean relative intensity of the C×43 band, normalized to vimentin or GAPDH, for the two groups was compared. Regardless of which protein was used as the internal standard, the mean relative C×43 level was significantly higher for samples taken from patients who became pregnant (P<0.01) (FIG. 4C). Correspondingly, for vimentin-normalized samples, the pregnancy rate in the higher C×43 group (more than the mean) was 57.1% while the pregnancy rate in the lower C×43 group (less than the mean) was only 28.2%. For GAPDH-normalized samples the corresponding difference was 71.9% versus 24.5%. These results indicate that C×43 level in follicular cells is one factor influencing pregnancy outcome after ICSI.

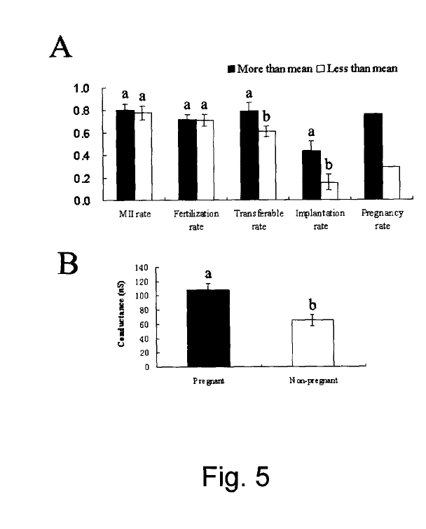

The relationship between gap junctional conductance, measured by single patch voltage clamp, and clinical outcome was also examined. FIG. 5A shows that, as with C×43 level, there was no relationship between conductance and either MII rate or fertilization rate, but high conductance (above the population mean) was positively associated with higher transferable embryo rate, implantation rate, and pregnancy rate. Correspondingly, follicular cells from patients who became pregnant after ICSI exhibited significantly higher gap junctional conductance (FIG. 5B).

### Discussion

In the present study, it was found that the C×43 level in follicular cells varies between patients, and that those patients whose follicular cells fell within the higher C×43-expressing group had higher transferable and implantation rates and were more likely to have a successful pregnancy outcome. Likewise, the mean relative C×43 level in follicular cells from pregnant patients was significantly higher than that in non-pregnant patients. Despite the fact that patient age is one of factors that affect pregnancy outcome from IVF, C×43 and conductance levels were not correlated with patient age in this study. Follicular cell C×43 can thus be added to the list of markers of oocyte and embryo developmental competence.

In conclusion, the present data indicate that C×43 is a major contributor to gap junctions in human follicular cells. C×43 level in follicular cells has a close relationship with intercellular coupling and pregnancy outcome, implicating it as a predictor of pregnancy outcome in assisted conception.

### Example 2

### Dye Transfer Assay for Gap Junctional Coupling

A dye transfer assay was used to determine gap junctional coupling strength among follicular cells. The strength of coupling can be obtained by determining the % of dye transfer, i.e. the number of cells receiving dye from the injected cell as a percentage of the total number of cells. The tendency of the dye to concentrate in the cell nuclei facilitates identification of individual cells.

A glass pipette with a tip of diameter 0.5-1.0 micrometer was filled to capacity with a 5% Lucifer yellow dye solution in distilled water. The dye was injected for 1 minute into a single cell of a follicular cell cluster obtained from a patient undergoing in vitro fertilization and the dye was allowed to spread to other cells for a further 1 minute before taking a photograph. Dye transfer order was determined by viewing with a fluorescence microscope.

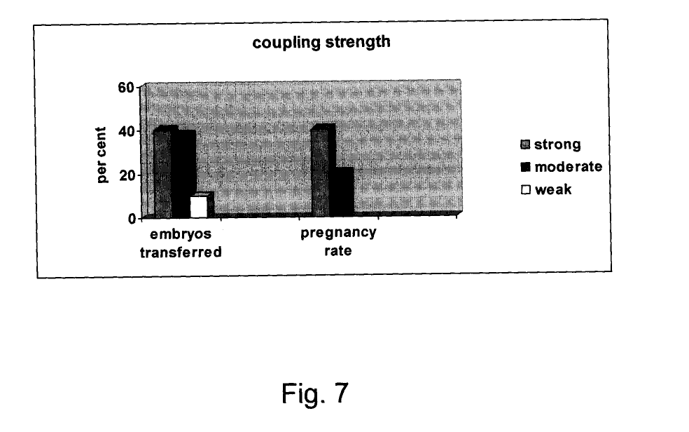

Data were obtained from 58 patients and showed (FIG. 7) that the mean coupling strength of individual follicles giving rise to transferred embryos correlates with the % embryos transferred (an indication of overall embryo quality) and the % pregnancy from that cohort of patients.

### Example 3

### Gap Junctional Coupling Strength

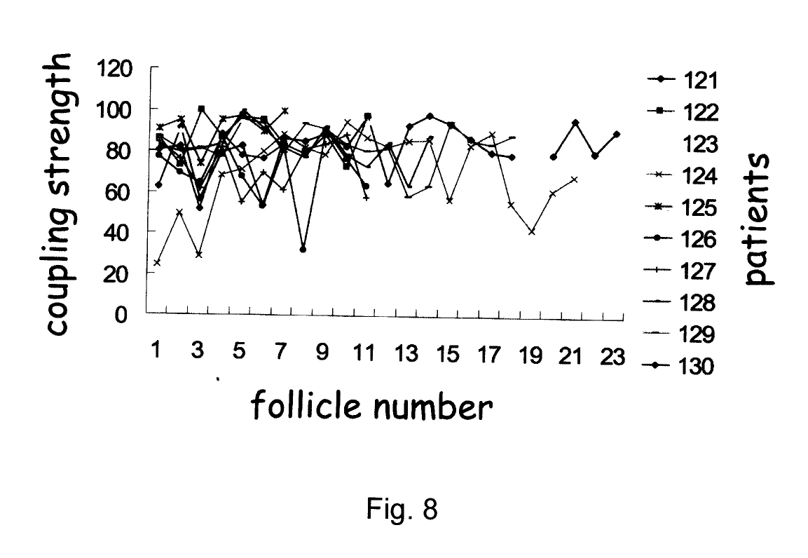

Using dye transfer as described in Example 2, the coupling strength of multiple follicles of individual patients was determined. The results are illustrated in FIG. 8, in which it is evident that individual follicles from the same patient have different coupling scores. In addition, FIG. 9 provides a summary of the relationship between mean coupling strength of follicles that gave rise to transferred embryos and pregnancy rate. At least two embryos were transferred to each patient.

### REFERENCES

1. Kidder G M. Roles of gap junctions in ovarian folliculogenesis: implications for female infertility. In: Winterhager E, editor. Gap Junctions in Development and Disease. Heidelberg, Germany: Springer Verlag; 2005. p. 223-37.

2. Tong D, Gittens J E, Kidder G M, Bai D. Patch-clamp study reveals that the importance of connexin43-mediated gap junctional communication for ovarian folliculogenesis is strain specific in the mouse. Am J Physiol Cell Physiol. 2006; 290: C290-7.

1. A method of assessing the health of each oocyte, or an embryo that may result therefrom, from a plurality of oocytes of a mammal, comprising the steps of: i) measuring gap junctional coupling strength of follicular cells associated with each oocyte; and ii) identifying the gap junctional coupling strength associated with each oocyte, wherein the greater the coupling strength of an oocyte relative to the coupling strength of each other oocyte, the healthier the oocyte and the embryo resulting from that oocyte.

2. A method as defined in claim 1, gap junctional coupling strength of follicular cells is measured by: i) culturing a sample of follicular cells associated with each of said independent oocytes; ii) injecting a dye into a single cell of each sample of follicular cells; and iii) determining the extent of dye transfer for each sample of follicular cells, wherein the greater the dye transfer, the greater the coupling strength.

3. A method as defined in claim 2, wherein the extent of dye transfer is based on the percentage of cells containing dye following injection.

4. A method as defined in claim 2, wherein the extent of dye transfer is based on the number of cells away from the single injected cell that contain dye.

5. A method as defined in claim 1, wherein gap junctional coupling strength is determined by measuring the electrical conductance between the cells of each follicle, wherein the greater the current, the greater the coupling strength.

6. A method of assessing the health of each oocyte, or an embryo that may result therefrom, from a plurality of oocytes of a mammal, comprising the step of determining the expression level of connexin43 in a sample of follicular cells associated with each oocyte, wherein the greater the expression level of connexin43 in a sample of follicular cells relative to the connexin43 expression level obtained for each other sample of follicular cells, the healthier the oocyte and the embryo that may result therefrom.

7. The method of claim 1 for use in determining the health of embryonic stem cells, wherein the healthier an embryo relative to other embryos of a plurality of embryos, the healthier the embryonic stem cells arising therefrom.

8. The method of claim 6 for use in determining the health of embryonic stem cells, wherein the healthier an embryo relative to other embryos of a plurality of embryos, the healthier the embryonic stem cells arising therefrom.

9. A kit useful to conduct the method of claim 1 comprising:
i) a dye-delivery means adapted to deliver a controlled amount of dye to a single follicular cell of a sample of follicular cells associated with an oocyte or embryo, wherein the dye-delivery means includes a suitable dye for injection into the follicular cell; and
ii) an indication that the greater the dye uptake of a sample of follicular cells associated with an oocyte/embryo of a mammal relative to the dye uptake of samples of follicular cells associated with other oocytes/embryos for the mammal, the healthier the oocyte or embryo.

10. A kit as defined in claim 9, wherein the dye delivery means is a micropipette.

11. A kit as defined in claim 10, wherein the micropipette has a beveled tip with a diameter in the range of about 0.5 to 1.0 μm.

12. A kit as defined in claim 9, wherein the dye delivery means includes a dye solution comprising about 1-5% dye.

13. A kit useful to conduct the method of claim 6 comprising:
i) a solid support to which is bound a first connexin43-specific reactant useful to immobilize connexin43 thereto from a follicular cell sample; and
ii) an indicator suitable for use to detect bound connexin43.

14. A kit as defined in claim 13, wherein the indicator comprises a second connexin43-specific reactant and a detectable label.

15. A kit as defined in claim 14, wherein the first and second connexin43-specific reactants are antibodies to connexin43.

16. A kit as defined in claim 14, wherein the detectable label is an enzyme.

17. A kit as defined in claim 16, wherein the enzyme is selected from the group consisting of horseradish peroxidase (HRP), alkaline phosphatase (AP), β-galactosidase, acetylcholinesterase and catalase.

18. A kit as defined in claim 16, additionally comprising a substrate for the enzyme.

19. The method of claim 1 wherein oocytes of the plurality are from different like mammals.

20. The method of claim 6 wherein oocytes of the plurality are from different like mammals.

21. A method as defined in claim 6, comprising the additional step of determining the expression level of at least one other follicular cell protein.
